(19) United States
(12) Patent Application Publication
(10) Pub. No.: US 2018/0236119 A1
(43) Pub. Date: Aug. 23, 2018

(54) WEARABLE FRAGRANCE EMISSION DEVICE

(71) Applicant: Trackmind Solutions LLC, New Brunswick, NJ (US)

(72) Inventor: Siddharth Shah, Freehold, NJ (US)

(21) Appl. No.: 15/923,137

(22) Filed: Mar. 16, 2018

Related U.S. Application Data
(63) Continuation of application No. 14/337,685, filed on Jul. 22, 2014.

(51) Int. Cl. A61L 2/24; C11B 9/00; A61L 2/00

(52) U.S. Cl. CPC A61L 2/24; A61L 2/0082; C11B 9/00

(57) ABSTRACT

A method and apparatus are provided to determine a pattern of emitting scents uniquely tailored to a particular user. A wearable fragrance emission device may store a fragrance emission pattern which correlates particular conditions with scents to be emitted from the device. The device detects conditions, such as environmental conditions or conditions related to the user, and periodically emits one or more fragrances in response to detected conditions. The wearable fragrance emission device may include one or more sensors for detecting the conditions.

### BACKGROUND

Many people wear perfume or cologne daily. However, these scents wear off after a period of time. Moreover, only one scent can be worn for that period of time. Further, people are typically limited in their selection of the scent. For example, because of the high cost of many perfumes and colognes, people usually select one scent and purchase a bottle, and use the bottle until it runs out. This can be months or years until the bottle runs out, during which time the user is limited to that scent.

### SUMMARY

One aspect of the present invention provides a wearable fragrance emission device, comprising a base adapted to be attached to at least one of an article of clothing or skin, a plurality of heat reaction elements residing on the base, a plurality of fragrance cartridges positioned in association with the plurality of heat reaction elements. The device may further include circuitry positioned on the base and in communication with a mobile device and the plurality of heat reaction elements, the circuitry programmed to determine whether one or more conditions are met, and when the one or more conditions are met, trigger selected ones of the plurality of heat reaction elements according to an emission pattern, the emission pattern generated based on user input. Determining whether one or more conditions are met may include receiving signals from a mobile device in communication with the wearable fragrance emission device. The device itself may be a patch or other structure. The fragrance cartridges may include at least one of paper, oils, beads, tubes, and wax. A housing may encase at least the plurality of fragrance cartridges, and potentially other elements of the device. For example, the housing may include a structure corresponding to a shape of the fragrance cartridges and adapted to hold the fragrance cartridges in place. In some examples, the device may also include one or more sensors for detecting a condition. Alternatively or additionally, the device may include a transmitter and receiver adapted to wirelessly exchange information with a mobile device.

Another aspect of the invention provides a method of emitting fragrances from a wearable fragrance emission device. The method includes receiving a fragrance emission pattern identifying one or more scents to be emitted in given quantities at given times, detecting one or more environmental conditions, comparing the detected environmental conditions to the fragrance emission pattern, and triggering emission of selected fragrances according to the fragrance emission pattern. The detected environmental conditions may include at least one of weather, location, time, and body temperature of a user of the wearable fragrance emission device. Triggering emission of the selected fragrances may include activating selected heating elements corresponding to the selected fragrances. Detecting the one or more environmental conditions can comprise receiving signals from a mobile device in communication with the wearable fragrance emission device.

In some examples, the method further includes detecting a level of fragrance remaining in one or more fragrance cartridges included on the wearable fragrance emission device, comparing the detected level to a predetermined threshold, and if the detected level falls below the predetermined threshold, automatically transmitting a request for a refill cartridge. Automatically transmitting the request for a refill may include communicating with a mobile device, such communicating causing the mobile device to complete a transaction over a network.

A further aspect of the invention provides a method comprising receiving, with one or more processors, first user input relating to user habits and preferences, generating, with one or more processors, a scent profile based on the first user input, generating, with one or more processors, a fragrance emission pattern based on at least one of the first user input and the scent profile, the emission pattern identifying one or more scents to be emitted in given quantities at given times, receiving, with one or more processors, second user input related to the fragrance emission pattern, and updating, with one or more processors, at least one of the scent profile and the fragrance emission pattern based on the second user input. Generating the fragrance emission pattern may include determining, based on the first user input, at least one condition for emitting scents and determining, based on the first user input, at least one scent to be emitted.

### BRIEF DESCRIPTION OF THE DRAWINGS

FIG. 1 is an example system diagram.

FIG. 2 illustrates an example user interface for providing first user input.

FIG. 3 illustrates an example user interface for providing second user input.

FIG. 4A is a cutaway sideview of an example wearable fragrance emission device.

FIG. 4B is a top view of another example wearable fragrance emission device.

FIG. 5 is a cutaway sideview of another example wearable fragrance emission device.

FIG. 6 is a cutaway sideview of another example wearable fragrance emission device.

FIGS. 7A-7B are cutaway sideviews of another example wearable fragrance emission device.

FIG. 8 is a cutaway sideview of another example wearable fragrance emission device.

FIG. 9 is a flow diagram of an example method.

### DETAILED DESCRIPTION

The disclosure is directed to a wearable fragrance emission device and a system and method for emitting fragrances in response to user preferences and environmental conditions.

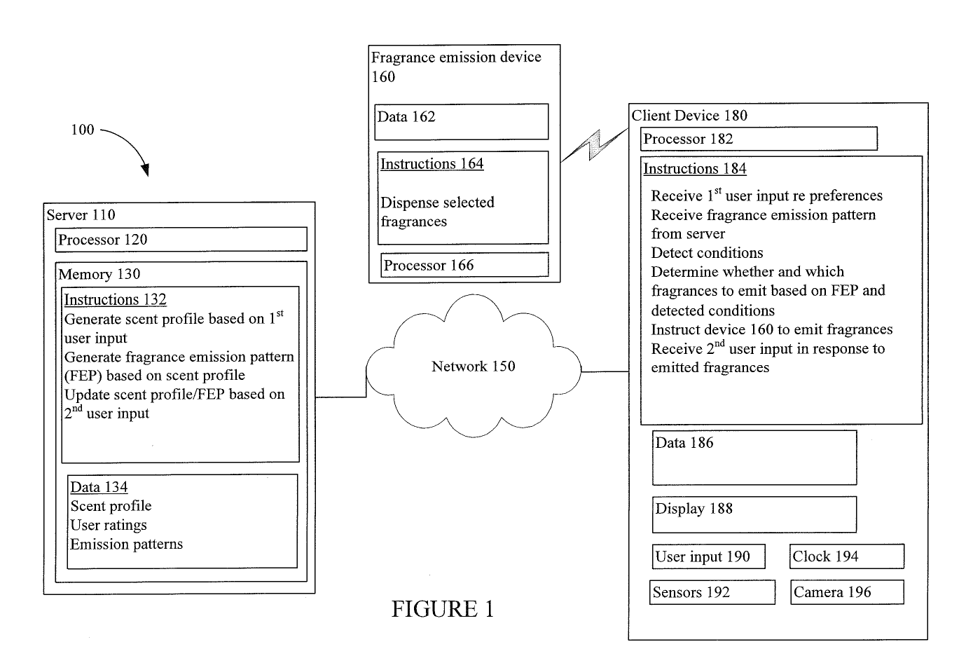

As shown in FIG. 1, an exemplary system 100 may include computers 110, 160, and 180. Computer 110 may be, for example, a server and may contain a processor 120, memory 130 and other components typically present in general purpose computers. Computer 180 may be a client device in wireless communication with fragrance emission device 160.

Memory 130 of computer 110 stores information accessible by processor 120, including instructions 132 that may be executed by the processor 120. Memory also includes data 134 that may be retrieved, manipulated or stored by the processor. The memory may be of any type capable of storing information accessible by the processor, such as a hard-drive, memory card, ROM, RAM, DVD, CD-ROM, write-capable, and read-only memories. The processor 120 may be any well-known processor or a dedicated controller, such as an ASIC.

The instructions 132 may be any set of instructions to be executed directly (such as machine code) or indirectly (such as scripts) by the processor. In that regard, the terms “instructions,” “steps” and “programs” may be used interchangeably herein. The instructions may be stored in object code format for direct processing by the processor, or in any other computer language including scripts or collections of independent source code modules that are interpreted on demand or compiled in advance. As described in further detail below, the instructions 132 may be executed to generate a scent profile based on first user input, generate a fragrance emission pattern based on at least one of the first user input and the scent profile, and update the scent profile and/or the first user input based on second user input.

Data 134 may be retrieved, stored or modified by processor 120 in accordance with the instructions 132. For instance, although the system and method is not limited by any particular data structure, the data may be stored in computer registers, in a relational database as a table having a plurality of different fields and records, or XML documents. The data may also be formatted in any computer-readable format such as, but not limited to, binary values, ASCII or Unicode. Moreover, the data may comprise any information sufficient to identify the relevant information, such as numbers, descriptive text, proprietary codes, pointers, references to data stored in other memories (including other network locations) or information that is used by a function to calculate the relevant data.

The data 134 may include, for example, one or more user scent profiles, user ratings associated with each scent profile, and fragrance emission patterns associated with each scent profile.

The scent profile may include, for example, user preferences in association with user information. For example, the user scent profile may include a user name and general user information (e.g., age, sex, background, etc.), the first user input, identified scent preferences, scents selected for inclusion in the user's wearable fragrance emission device, a quantity of remaining fragrance in the emission device, and any other information.

The fragrance emission pattern may be generated based on the user scent profile and/or the first user input. The fragrance emission pattern may resemble a schedule for emitting selected fragrances, and may further include conditions for emitting selected fragrances. For example, the fragrance emission pattern may be one or more algorithms causing emission of one or more fragrances in response to detecting one or more environmental conditions, such as weather, time, location, etc.

The user ratings may include feedback provided by users in response to scents being emitted from the fragrance emission device 160. For example, user may rate the scents themselves, strengths of the scents, frequency with which the scent is emitted, or any of a variety of other characteristics.

Although FIG. 1 functionally illustrates the processor and memory as being within the same block, it will be understood by those of ordinary skill in the art that the processor and memory may actually comprise multiple processors and memories that may or may not be stored within the same physical housing. For example, some of the instructions and data may be stored on removable CD-ROM and others within a read-only computer chip. Some or all of the instructions and data may be stored in a location physically remote from, yet still accessible by, the processor. Similarly, the processor may actually comprise a collection of processors which may or may not operate in parallel.

The computer 110 may be at one node of a network 150 and capable of directly and indirectly communicating with other nodes of the network. For example, computer 110 may comprise a web server that is capable of communicating with client device 180 via network 150 such that server 110 uses network 150 to transmit and display information to a user on display of client device 180.

Each client device may be configured similarly to the server 110, with a processor 182 and memory including instructions 184 and data 186. Each client computer 180 may be a personal computer having all the internal components normally found in a personal computer such as a central processing unit (CPU), display device 188 (for example, a monitor having a screen, a projector, a touch-screen, a small LCD screen, a television, or another device such as an electrical device that is operable to display information processed by the processor), CD-ROM, hard-drive, user input 190 (for example, a mouse, keyboard, touch-screen or microphone), speakers, modem and/or network interface device (telephone, cable or otherwise) and all of the components used for connecting these elements to one another. Moreover, computers in accordance with the systems and methods described herein may comprise any device capable of processing instructions and transmitting data to and from humans and other computers including general purpose computers, PDAs, network computers lacking local storage capability, set-top boxes for televisions, and other networked devices.

Although the computer 180 may comprise a full-sized personal computer, the system and method may also be used in connection with mobile devices capable of wirelessly exchanging data with a server over a network such as the Internet. By way of example only, client device 180 may be a wireless-enabled PDA, hand-held navigation device, tablet PC, netbook, or a cellular phone capable of obtaining information via the Internet. The user may input information, for example, using a small keyboard, a keypad, or a touch screen.

The server 110 and client computer 180 are capable of direct and indirect communication, such as over network 150. Although only a few computers are depicted in FIG. 1, it should be appreciated that a typical system can include a large number of connected computers, with each different computer being at a different node of the network 150. The network, and intervening nodes, may comprise various configurations and protocols including the Internet, World Wide Web, intranets, virtual private networks, wide area networks, local networks, private networks using communication protocols proprietary to one or more companies, Ethernet, WiFi (such as 802.11, 802.11b, g, n, or other such standards), and HTTP, and various combinations of the foregoing. Such communication may be facilitated by any device capable of transmitting data to and from other computers, such as modems (e.g., dial-up, cable or fiber optic) and wireless interfaces.

Although certain advantages are obtained when information is transmitted or received as noted above, other aspects of the system and method are not limited to any particular manner of transmission of information. For example, in some aspects, information may be sent via a medium such as a disk, tape or CD-ROM. Yet further, although some functions are indicated as taking place on a single client device having a single processor, various aspects of the system and method may be implemented by a plurality of computers, for example, communicating information over network 150.

The data 186 of the client device 180 may store a local copy of the fragrance emission pattern. The instructions 184 may include instructions for receiving first user input relating to user habits, preferences, etc., receiving a fragrance emission pattern from the server 110, detecting environmental and other conditions and determining whether and which fragrances should be emitted in response thereto, instructing the fragrance emission device 160 to emit the determined fragrance, and receiving second user input in response to the emitted fragrance.

The client device 180 may also include one or more sensors 192, a clock 194, a camera 196, and other elements typically found in mobile devices or other computing devices. The sensors 192 may include any of a variety of sensors, such as light sensors, motion sensors, thermometers, heart rate monitors, pedometers, accelerometers, gyroscopes, global positioning satellite (GPS) receiver, etc. These sensors 192 may be used to detect various environmental conditions, including location, weather, or the like. The clock may be used to determine a day and time. In response to the detected environmental conditions and/or day/time, the client device 180 may determine that a particular scent should be emitted from the wearable device 160, and may instruct the wearable device to emit such fragrance. The camera 196 may also be used to detect environmental conditions. By way of example only, the camera 196 may capture an image of a location in which the user is. Object detection algorithms may be used to identify objects or conditions in the image. In turn, the client device may determine that particular fragrances should be emitted in response to those conditions. For example, if the captured image is determined to include a number of animals, the client device may instruct the fragrance emission device to release a scent or combination of scents to cover up, counteract, or complement a smell from the animals.

Fragrance emission device 160, similar to the server 110 and client 180, may include data 162, instructions 164, and processor 166. The instructions 164 may provide for emitting selected fragrances. The processor 166, in some examples, may be a microprocessor, a state machine, or any other type of processor. The fragrance emission device 160 may be a wearable device, such as a patch that may be adhered to a user's skin or clothes. The device may be disposable, or may be refilled when the fragrances run out. In other examples the device 160 may be another type of device, such as an air freshener pluggable into an outlet, a free-standing air freshener, or the like.

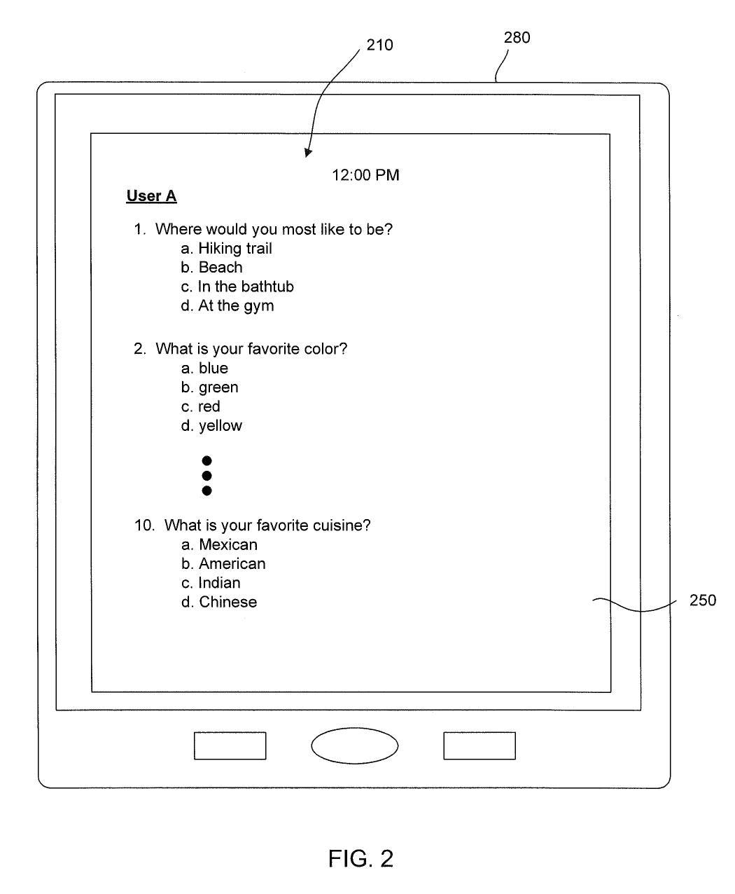

FIG. 2 illustrates an example user interface 210 of a client device for receiving first input from a user. While in this example the user interface 210 is included in a mobile device 280, it should be understood that the user interface may be included in any type of client device, such as those described above in connection with FIG. 1.

The first input provides information regarding user habits, preferences, and the like. For example, the user input may include lifestyle information, such as types of activities the user engages in, types of food the user enjoys, a schedule the user typically follows, the user's occupation, where the user lives, or any other type of information relating to the user's life. The first user input may also include scent preferences, such as vanilla, lavender, pine, etc., and intensity preferences, such as whether the user prefers strong or mild fragrances. In some examples, the first user input may also include information regarding the user's past fragrance usage, such as which scents they have used and whether they liked those scents.

According to one example, the user may be prompted to enter the first input by a series of questions, such as multiple choice questions, yes/no questions, free-answer questions, or ratings questions. For example, the user may be presented with a survey, such as the survey shown on display 250 of the mobile device 280. In some examples, questions presented to the user may vary based on the user's response to previous questions.

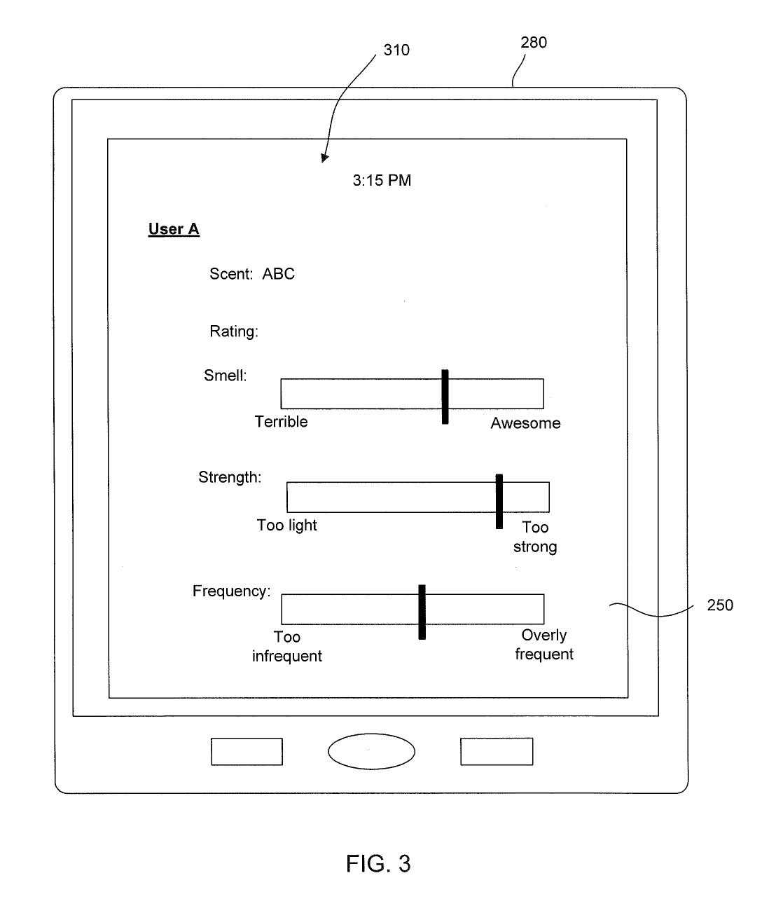

FIG. 3 illustrates another example user interface 310, for example providing second user input. The second user input may be provided in response to fragrances emitted by the wearable fragrance emission device 160 of FIG. 1. The second user input may also be provided via the display 250 of the mobile device 280. The information provided in the second user input may include, for example, ratings of the scents emitted. The ratings may address the scents themselves, the strength of the scent, the frequency of emission of the scents, etc. As shown in FIG. 3, the second input may be entered using a sliding scale. According to other examples, however, the input may be entered using a 5-star or 10-star rating system, a numeric scale, questions and answers, free text or speech, or any other mechanism.

As mentioned above, the second user input may be used to update the user profile and/or fragrance emission pattern generated for the user. According to some examples, the user may periodically enter the second user input, either voluntarily or when prompted, in order to continually fine-tune the fragrances emitted by the wearable device. As such, after a period of time, such as a few days or weeks, the system creates a pattern of emitting scents that are uniquely tailored and optimally desirable to the user.

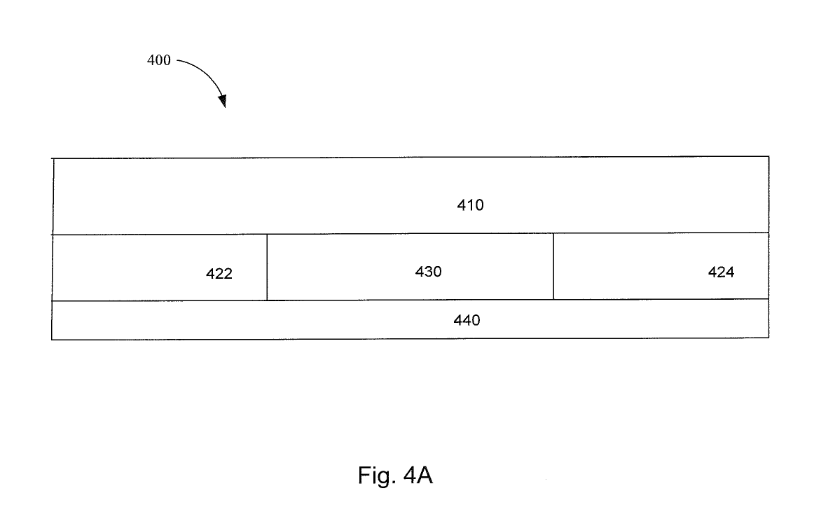

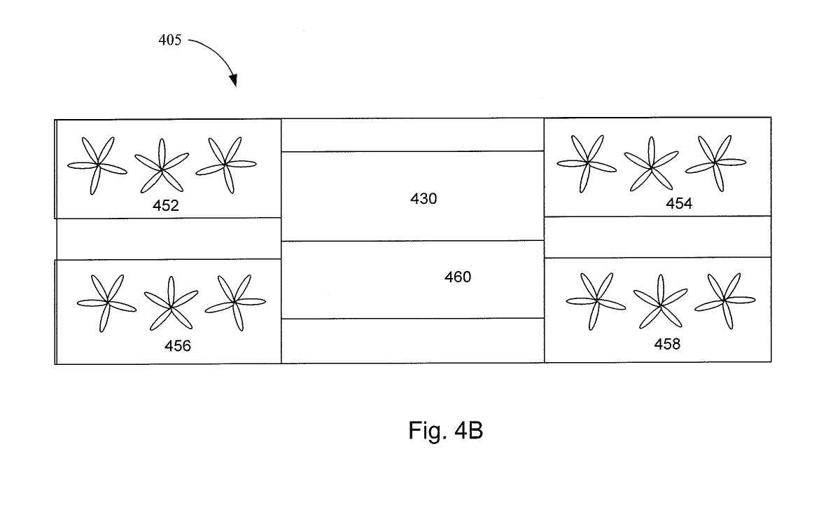

FIGS. 4-8 illustrate examples of wearable fragrance emission devices. As shown in FIG. 4A, device 400 includes a fragrance layer 410 as an upper layer, an adhesive layer 440 as a bottom layer, and a computer chip 430 and one or more heating elements 422, 424 sandwiched between the upper layer and bottom layer.

The fragrance layer 410 may include one or more fragrance tubes, beads, wax cubes, oil, scented paper, or any combination of these or other fragrance elements. For example, the fragrance emission device 400 may include a housing having a cavity in which the fragrance elements reside. One or more surfaces or areas of the housing may be porous, thereby enabling the fragrance to escape from the housing. Moreover, the housing may in some examples be refilled with fragrance elements, such as by being removed, refilled, and reattached.

The adhesive layer 440 may include, for example, a thin layer of material having a sticky substance applied thereon. In this regard, the user may peel away a protective layer from the device, thereby exposing the adhesive layer 440 for application to the user's skin, clothing, etc. In other examples, the adhesive layer may include one or more fasteners, such as hook-and-loop fasteners, clips, or any other type of fastener.

The computer chip 430 may be, for example, a microchip including a processor, such as a microprocessor or a state machine, and a memory. As such, the computer chip 430 may be used to detect ambient conditions, and determine a response of the wearable fragrance emission device 400. Example conditions detected by the computer chip 430 include temperature, date, time, location, odors, etc. In this regard, the wearable fragrance emission device may also include a thermometer, clock, location device (e.g., global positioning satellite (GPS), compass, gyroscope, etc.), light sensor, sniffer, or any of a variety of other sensors. In some examples, the wearable fragrance emission device 400 may also detect an activity level of the user wearing the device 400. For example, the device 400 may include a motion sensor, pedometer, heart-rate sensor, or the like.

The one or more heating elements 422, 424 may be positioned near and communicatively coupled to the computer chip 430, such that the computer chip 420 activates the heating elements 422, 424 in response to detecting one or more conditions. For example, upon determining that it is currently a particular time of day or temperature, the computer chip 430 may transmit a signal to the one or more heating elements 422, 424 causing them to activate. Activation of the heating elements 422, 424 may in turn trigger release of the fragrance in the fragrance layer 410.

While only two heating elements are shown, it should be understood that the device 400 may include more or fewer heating elements. Moreover, the fragrance layer 410 may include multiple different scents. The scents may be positioned corresponding to the heating elements 422, 424, such that particular heating elements may be triggered at a given time, thereby selectively releasing particular fragrances.

Although not shown, in some examples the wearable fragrance emission device 400 may also include a power supply, such as a battery. The power supply may be used to power the computer chip 430, the one or more heating elements 422, 424, or any other elements of the device 400.

According to some aspects, the computer chip 430 and/or one or more heating elements 422, 424 may be omitted from the device 400. For example, emission of the fragrance from the fragrance layer 410 may be triggered by heat emanating from the user. For instance, if the user is engaging in strenuous physical activity, the user's body temperature may rise. The increased body temperature may activate release of the fragrance from the fragrance layer 410.

Moreover, the fragrance in the fragrance layer 410 may be released via another mechanism. For example, as shown in FIG. 4B, one or more micro fans 452, 454, 456, 458 may be coupled to the computer chip 430 and selectively activated to release scent from a fragrance layer above the fans. For example, the fans 452-458 may be coupled to a micro motor 460. As one or more of the fans 452-458 are activated, they may push air through the fragrance layer, the air carrying a correspondingly positioned fragrance from the fragrance layer. In this example, air intake channels in the fan layer may be provided, and a material encasing the fragrance layer may be porous thereby enabling the air to the pushed therethrough.

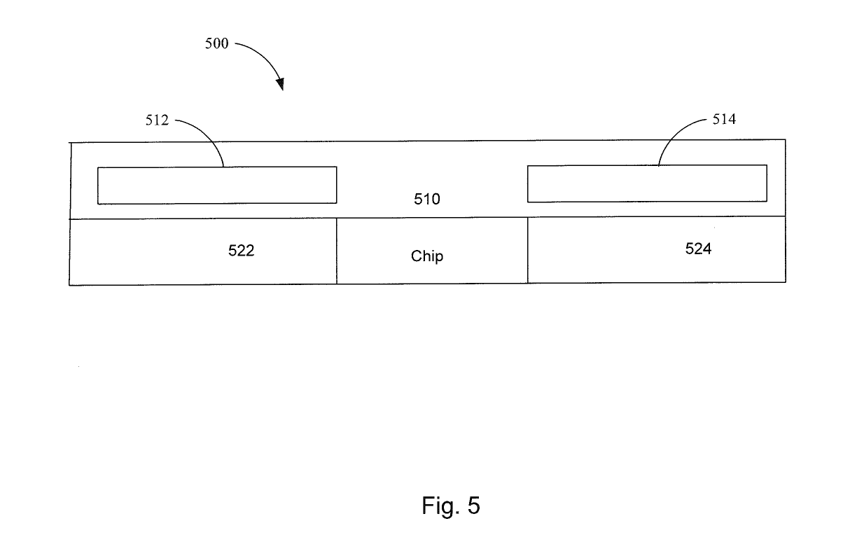

FIG. 5 illustrates another example fragrance emission device 500. The fragrance emission device 500 includes many of the same elements as the fragrance emission device 400 described above in connection with FIG. 4. However, as shown, the fragrance emission device 500 includes one or more replaceable fragrance tubes 512, 514 in fragrance layer 510. The replaceable fragrance tubes 512, 514 may be any shape, such as cylindrical, cubical, rectangular, spherical, etc. The tubes 512, 514 may be held in place by a corresponding structure, such as a housing encasing the fragrance layer having a complementary shaped cutout for the tubes 512, 514. Accordingly, the tubes 512, 514 may be removed and reinserted through a cutout, opening, or door in the housing.

The replaceable fragrance tubes 512, 514 may be positioned in relation to heating elements 522, 524. For example, the fragrance tube 512 may be positioned above a first heating element 522, while the fragrance tube 514 is positioned above a second heating elements 524. Moreover, each fragrance tubes 512, 514 may be filled with a different scent. In this regard, one or more fragrance tubes 512, 514 may release a fragrance as a result of activating heating elements 522, 524 at a given time. For example, the heating element 522 may be activated to release a first scent from fragrance tube 512, the heating element 524 may be activated to release a second scent from tube 514, or both heating elements 522, 524 may be simultaneously activated to release a third scent, which is a combination of the scents in tubes 512 and 514. While two fragrance tubes 512, 514 are shown in FIG. 5, it should be understood that any number of tubes and corresponding heating elements may be included.

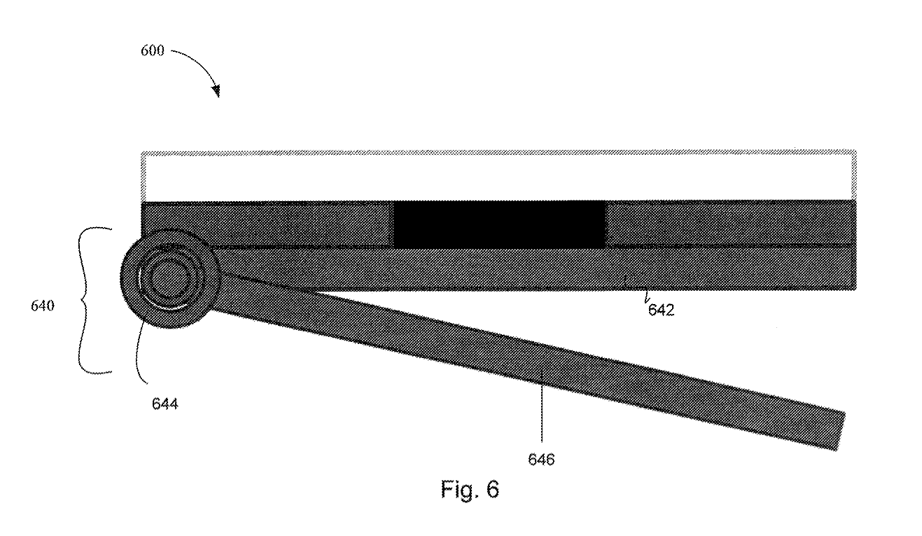

FIG. 6 illustrates another example wearable fragrance emission device 600. In this example, the device 600 may be secured to an article of clothing or other object using a clip 640. The clip 640 may include a base 642 supporting the device 600, and an articulating arm 646 coupled to the base 642 via a spring 644.

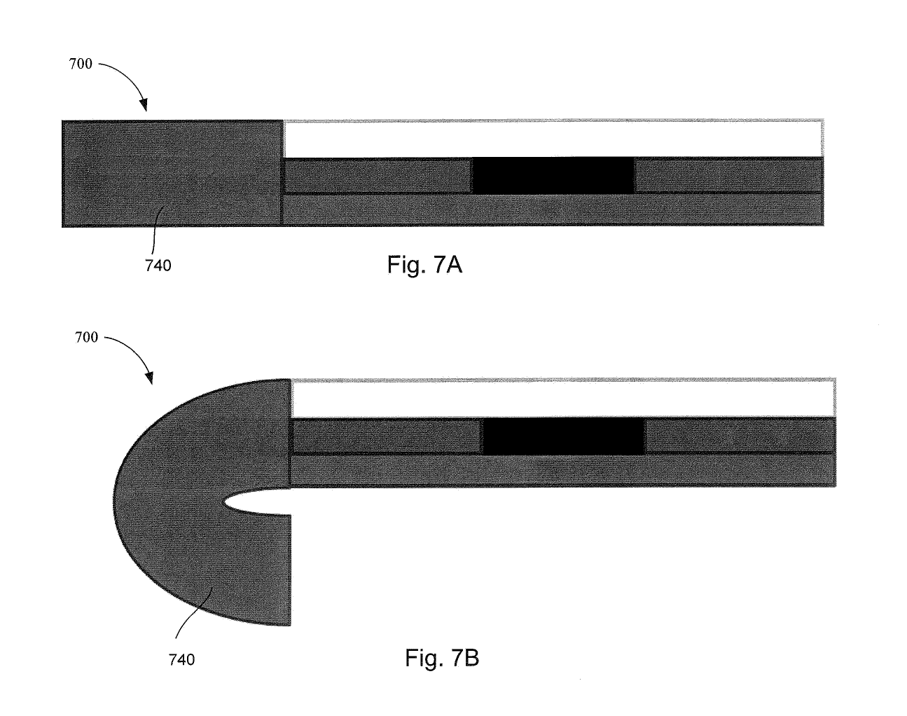

FIGS. 7A-B illustrate yet another example wearable fragrance emission device 700. In this example, the device 700 includes a flexible clip 740 which may be used to secure the device 700. For example, the flexible clip 740 may include a deformable material, such as metal, plastic, or other material, that may be bent as shown in FIG. 7B. In this regard, the clip 740 may be positioned and secured around an object, such as an article of clothing. To remove the device 700, the clip 740 may return to its original shape as shown in FIG. 7A.

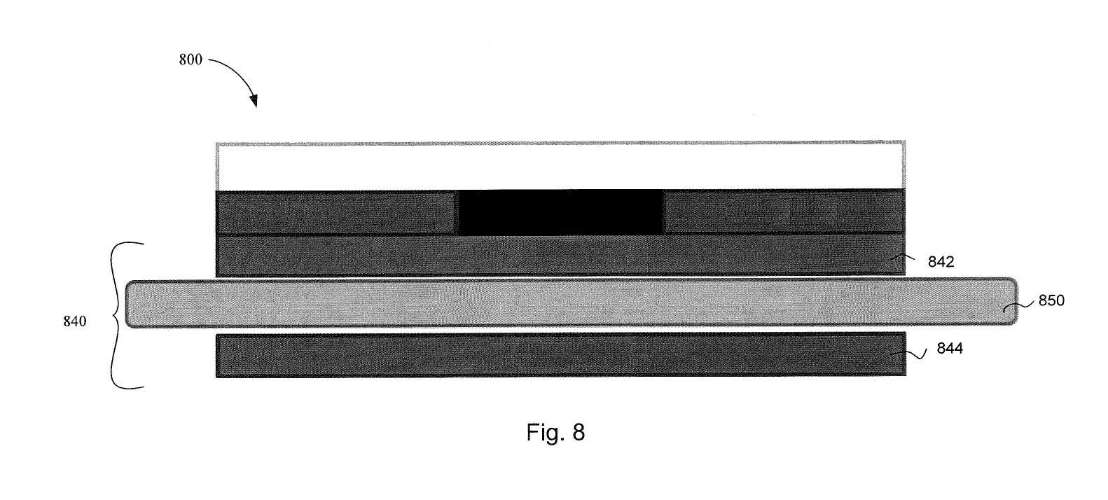

FIG. 8 illustrates another example wearable fragrance emission device 800. In this example, the device 800 includes a magnetic fastener 840. The magnetic fastener 840 includes a base 842 supporting the device 800, and a back 844 which mates with the base 842. For example the base 842 and back 844 may have opposite polarities, and therefore attract one another. As such, the magnetic fastener 840 may be used to secure the device 800 to a layer of clothing 850 by positioning the base 842 and back 844 on opposing sides of the layer of clothing 850.

While the examples above provide various possibilities for the housing and securing structure of the wearable fragrance emission device, it should be understood that various modifications may be made. For example, a size, shape, and material of the housing may be varied. Moreover, an arrangement of the elements within the housing may be varied, such as by moving the computer chip or other electronic circuitry with respect to the fragrance emission elements or heating elements.

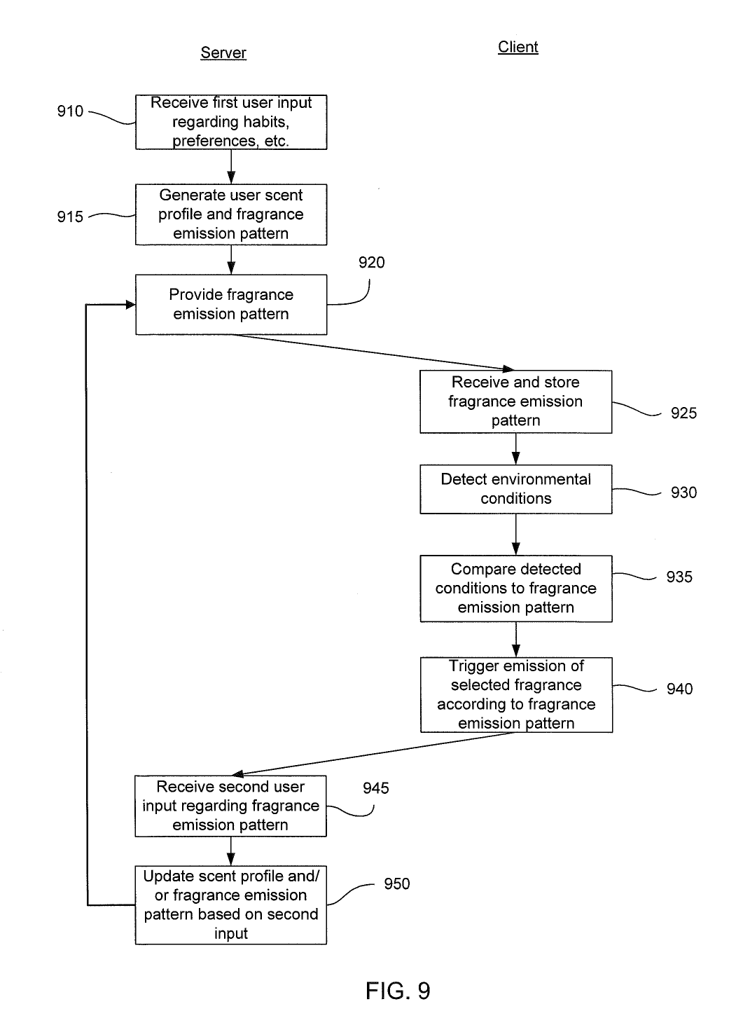

FIG. 9 provides an example flow diagram illustrating a method 900 for emitting fragrances uniquely tailored to a user's preferences. The method may be performed by, for example, a server computing device in direct or indirect communication with a wearable fragrance emission device. In other examples, the entire method 900 may be performed by one device, such as by an integrated fragrance emission and computing device. It should be understood that the operations involved in the below methods need not be performed in the precise order described. Rather, various operations may be handled in a different order or simultaneously, and operations may be added or omitted.

In block 910, first user input is received. The first user input may relate to habits, preferences, and other information relating to the user. The input may be received at the server computing device, for example, via transmission by a client computing device.

In block 915, a user scent profile and a fragrance emission pattern are generated based on the first user input. The scent profile may include, for example, user preferences in association with user information. For example, the user scent profile may include a user name and general user information (e.g., age, sex, background, etc.), the first user input, identified scent preferences, scents selected for inclusion in the user's wearable fragrance emission device, a quantity of remaining fragrance in the emission device, and any other information. The fragrance emission pattern may be generated based on the user scent profile and/or the first user input. The fragrance emission pattern may resemble a schedule for emitting selected fragrances, and may further include conditions for emitting selected fragrances. For example, the fragrance emission pattern may be one or more algorithms causing emission of one or more fragrances in response to detecting one or more environmental conditions, such as weather, time, location, etc.

In block 920 the fragrance emission pattern is transmitted to the wearable fragrance emission device. For example, the pattern may be transmitted to a client device, and further downloaded onto the wearable device. In other examples, the pattern may be stored at the client device, such as a mobile smartphone, which maintains continual communication with the wearable device. In this regard, the client device may perform detection and computations, and may instruct the wearable device when to emit particular fragrances. The pattern is received and stored in block 925.

In block 930, environmental conditions are detected, for example, by the client device or wearable fragrance emission device. Such environmental conditions may include, for example, time, day, weather, temperature, heart rate, etc., and may be detected using any of a variety of sensors.

In block 935, the detected conditions are compared to the fragrance emission pattern, for example, to determine whether to emit a fragrance, and which fragrance to emit at which intensity.

In block 940, emission of selected fragrances is triggered according to the fragrance emission pattern. For example, the client device may transmit a signal to the wearable fragrance emission device to activate one or more heating elements corresponding to the selected fragrances.

In block 945, second user input is received regarding the fragrance emission pattern. The second user input may include evaluations or ratings of the emitted fragrances, including the scents, intensity, frequency, etc. of the emissions. The second user input may be received at the server computing device, and used to update the user scent profile and/or fragrance emission pattern (block 950). The process may then return to block 920.

While not shown in FIG. 9, in some examples the wearable fragrance emission device and/or client computing device may detect a remaining level of one or more fragrances in the wearable emission device. Accordingly, such devices may automatically order refills or replacements for the fragrances. For example, upon detecting that a remaining level of fragrance has fallen below a predetermined threshold, the client device may place an order with a fragrance supplier service to have the refills or replacements shipped to the user.

The systems and methods described above enable fragrances to be continually emitted for a user, rather than a perfume or cologne that wears off after a period of time. Moreover, the emitted scents may be varied based on one or more conditions, and further may be uniquely tailored to a user's preferences.

As these and other variations and combinations of the features discussed above can be utilized without departing from the subject matter as defined by the claims, the foregoing description of exemplary aspects should be taken by way of illustration rather than by way of limitation of the subject matter as defined by the claims. It will also be understood that the provision of the examples described herein (as well as clauses phrased as “such as,” “e.g.”, “including” and the like) should not be interpreted as limiting the claimed subject matter to the specific examples; rather, the examples are intended to illustrate only some of many possible aspects.

1. A method for emitting fragrances from a wearable fragrance emission device, the method comprising:
receiving, by one or more processors, first input relating to a user, the first input indicating user scent preferences;
generating, by the one or more processors, a fragrance emission pattern based on the first input, the fragrance emission pattern including conditions for emitting selected fragrances;
detecting, by one or more sensors in communication with the one or more processors, conditions surrounding the user at a given time;
determining, by the one or more processors, whether to emit a fragrance based on the detected conditions and the fragrance emission pattern;
selecting, by the one or more processors, one or more of a plurality of fragrances to emit based on the detected conditions and the fragrance emission pattern; and
activating emission of the selected one or more fragrances.

2. The method of claim 1, further comprising determining an intensity at which to emit the selected one or more fragrances.

3. The method of claim 1, wherein the conditions surrounding the user include environmental conditions.

4. The method of claim 3, wherein the conditions include at least one of weather, time, temperature, location, or surrounding objects.

5. The method of claim 1, wherein the conditions surrounding the user include body conditions of the user.

6. The method of claim 5, wherein the conditions include at least one of heart rate or body temperature.

7. The method of claim 1, further comprising:
receiving second input relating to the fragrance emission pattern, the second input including evaluations of the emitted fragrances; and
modifying the fragrance emission pattern based on the second input.

8. The method of claim 7, wherein the evaluations relate to the scent, intensity, and frequency of the emitted fragrances.

9. The method of claim 1, wherein the first input includes user lifestyle information, including at least one of: types of activities the user engages in, types of food the user enjoys, a schedule the user typically follows, the user's occupation, or where the user lives.

10. A wearable fragrance emission device, comprising:
a plurality of fragrances;
an emission activation mechanism corresponding to each of the plurality of fragrances;
a memory storing a fragrance emission pattern including conditions for emitting selected fragrances; and
one or more processors in communication with the memory and the emission activation mechanism, the one or more processors configured to:
detect conditions surrounding the wearable fragrance emission device at a given time;
determine whether to emit a fragrance based on the detected conditions and the fragrance emission pattern;
select one or more of the plurality of fragrances to emit based on the detected conditions and the fragrance emission pattern; and
trigger the emission activation mechanism corresponding to the selected one or more fragrances.

11. The device of claim 10, further comprising one or more sensors in communication with the one or more processors, the one or more sensors configured to detect the conditions surrounding the device.

12. The device of claim 11, wherein the one or more sensors include at least one of: a thermometer, location device, light sensor, or sniffer.

13. The device of claim 11, wherein the one or more sensors include at least one of a motion sensor, pedometer, or heart-rate sensor.

14. The device of claim 10, wherein the one or more processors are further configured to determine an intensity at which to emit the selected one or more fragrances.

15. The device of claim 1, wherein the fragrance emission pattern further includes a schedule for emitting particular fragrances at particular times.

16. A wearable fragrance emission device, comprising:
one or more fragrances;
an emission activation mechanism corresponding to each of the one or more fragrances;
one or more sensors;
one or more processors in communication with the one or more sensors and the emission activation mechanism, the one or more processors configured to:
receive input from the one or more sensors;
determine, based on the received input, conditions surrounding the wearable fragrance emission device at a given time;
determine whether to emit a given one of the one or more fragrances based on the detected conditions; and
trigger the emission activation mechanism corresponding to the given fragrance.

17. The device of claim 16, wherein the one or more sensors include a sniffer.

18. The device of claim 16, wherein the one or more sensors include a thermometer.

19. The device of claim 16, wherein the one or more sensors include a heart rate sensor.

20. The device of claim 16 wherein the one or more sensors include at least one of a location device, motion sensor, light sensor, or pedometer.
